(19) United States
(12) Patent Application Publication
(10) Pub. No.: US 2008/0077017 A1
(43) Pub. Date: Mar. 27, 2008

(54) ULTRASONIC PROBE, ULTRASONIC ENDOSCOPE, AND ULTRASONIC DIAGNOSTIC APPARATUS

(75) Inventor: Hiroaki HYUGA, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/861,101

(22) Filed: Sep. 25, 2007

(30) Foreign Application Priority Data
Sep. 26, 2006 (JP) 2006-260838

(51) Int. Cl. A61B 8/00

(52) U.S. Cl. 600/459

(57) ABSTRACT

In an ultrasonic probe to be used in an ultrasonic diagnostic apparatus for medical use, the temperature rise due to heat generated from ultrasonic transducers can be suppressed. The ultrasonic probe includes: an ultrasonic transducer array in which plural ultrasonic transducers are arranged with gaps in between; a casing for accommodating at least the ultrasonic transducer array; and channels for circulation of a liquid heat transfer material in the gaps between the plural ultrasonic transducers.

### BACKGROUND OF THE INVENTION

1. Field of the Invention

The present invention relates to an ultrasonic probe to be used when intracavitary scanning or extracavitary scanning is performed on an object to be inspected, and an ultrasonic endoscope to be inserted into a body cavity of the object. Further, the present invention relates to an ultrasonic diagnostic apparatus including such an ultrasonic probe or an ultrasonic endoscope and an ultrasonic diagnostic apparatus main body.

2. Description of a Related Art

In medical fields, various imaging technologies have been developed in order to observe the interior of an object to be inspected for making diagnoses. Especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but gynecology, circulatory system, digestive system, etc.

The ultrasonic imaging is an image generation technology utilizing the nature of ultrasonic waves that the ultrasonic waves are reflected at a boundary between regions with different acoustic impedances (e.g., a boundary between structures). Typically, an ultrasonic diagnostic apparatus (or referred to as an ultrasonic imaging apparatus or an ultrasonic observation apparatus) is provided with an ultrasonic probe to be used in contact with the object or ultrasonic probe to be inserted into a body cavity of the object. Alternatively, the apparatus may be provided with an ultrasonic endoscope in combination of an endoscope for optically observing the interior of the object and an ultrasonic probe for intracavitary.

Using such an ultrasonic probe or ultrasonic endoscope, ultrasonic beams are transmitted toward the object such as a human body and ultrasonic echoes generated by the object are received, and thereby, ultrasonic image information is acquired. On the basis of the ultrasonic image information, ultrasonic images of structures (e.g., internal organs, diseased tissues, or the like) existing within the object are displayed on a display unit of the ultrasonic diagnostic apparatus.

In the ultrasonic probe, a vibrator (piezoelectric vibrator) having electrodes formed on both sides of a material (a piezoelectric material) that expresses piezoelectric effect is generally used as an ultrasonic transducer for transmitting and receiving ultrasonic waves. As the piezoelectric material, a piezoelectric ceramics represented by PZT (Pb(lead) zirconate titanate), a polymeric piezoelectric material represented by PVDF (polyvinylidene difluoride), or the like is used.

When a voltage is applied to the electrodes of the vibrator, the piezoelectric material expands and contracts due to the piezoelectric effect to generate ultrasonic waves. Accordingly, plural vibrators are one-dimensionally or two-dimensionally arranged and the vibrators are sequentially driven, and thereby, an ultrasonic beam transmitted in a desired direction can be formed. Further, the vibrator receives the propagating ultrasonic waves, expands and contracts to generate an electric signal. The electric signal is used as a reception signal of ultrasonic waves.

When ultrasonic waves are transmitted, drive signals having great energy are supplied to the ultrasonic transducers. Not the whole energy of the drive signals is converted into acoustic energy and the considerable amount of energy turns into heat. Thus, there has been a problem of rising temperature of the ultrasonic probe during its use. However, the ultrasonic probe for medical use is used in direct contact with a living body of human or the like, and the surface temperature of the ultrasonic probe is requested to be 43° C. or below for safety reasons of prevention of low-temperature burn.

As a related technology, Japanese Patent Application Publication JP-P2002-291737A discloses an ultrasonic probe having an ultrasonic probe head part for transmitting and receiving ultrasonic waves, a cable electrically connected to the ultrasonic probe head part, and a cable cooling part thermally connected to at least one part of the cable.

However, in JP-P2002-291737A, only a small portion of the ultrasonic probe head part is indirectly cooled via the cable by cooling the cable, and therefore, the cooling efficiency is not so good.

Japanese Patent Application Publication JP-A-63-242246 discloses an ultrasonic probe for intracavitary to be inserted into a body cavity for imaging ultrasonic images, and the ultrasonic probe is provided with cooling means for cooling the heat generated by an ultrasonic converter during operation of the ultrasonic probe, in a predetermined position of a sound absorbing material. In JP-A-63-242246, a cooling pipe is provided in the ultrasonic probe and a cooling medium such as water is flown through the pipe, and thereby, a group of ultrasonic vibrators are cooled.

However, when the cooling pipe is provided on the side of the group of ultrasonic vibrators (FIG. 3), the thermal coupling between the cooling pipe and the group of ultrasonic vibrators becomes weaker and the cooling efficiency is not good. On the other hand, when the cooling pipe is provided on the back of the group of ultrasonic vibrators (FIGS. 4-6), there is a fear that the ultrasonic waves released to the back of the group of ultrasonic vibrators may not be sufficiently absorbed.

Japanese Patent Application Publication JP-A-11-299775 discloses an ultrasonic diagnostic apparatus including transferring means for guiding heat generated in a sound absorbing member to a position apart from the sound absorbing member, and releasing means provided at the position apart from the sound absorbing member, for releasing the heat guided by the transferring means. In the sound absorbing member, a surface opposite to a surface on which ultrasonic vibrators have been provided is formed in a curved configuration having a focus for reflecting and concentrating ultrasonic waves radiated from the ultrasonic vibrators toward the sound absorbing member, and a heat absorbing part of the transferring means is provided in the focus position within the sound absorbing member (FIG. 6).

In JP-A-11-299775, the temperature of the vibrator part at the leading end of an insertion part is controlled by electronic cooling means provided within the grip part of an ultrasonic probe via a heat pump (FIG. 5). Therefore, the vibrator part is indirectly cooled via the heat pump and so on, and therefore, the cooling efficiency is not good.

Japanese Patent Application Publication JP-A-61-58643 discloses an ultrasonic probe having ultrasonic vibrators and a case accommodating the vibrators, and the ultrasonic probe has means for guiding a cooling material to the object contact side of the ultrasonic vibrators.

However, when a cooling medium is flown along a front face of an acoustic lens, that is, through partition walls between the object contact surface and the acoustic lens of the ultrasonic probe (FIG. 1), the distance between the ultrasonic vibrators and the object becomes longer and causes attenuation of ultrasonic waves transmitted and received by the ultrasonic vibrators. On the other hand, when a channel for the cooling medium is provided within a back acoustic absorbing material (FIG. 3), there is a fear that the ultrasonic waves released to the back of ultrasonic vibrators may not be sufficiently absorbed. Further, when a channel for the cooling medium is provided between the back acoustic absorbing material and the case (FIG. 5), the thermal coupling between the ultrasonic vibrators and the cooling medium becomes weaker, and therefore, the cooling efficiency is not good.

Japanese Utility Model Application Publication JP-U-57-88073 discloses an ultrasonic probe provided with a path for a cooling medium in contact with the object outside of ultrasonic vibrators.

However, as shown in FIG. 1 of JP-U-57-88073, the path for the cooling medium is provided apart from the space where the ultrasonic vibrators are provided, and therefore, only the periphery of the ultrasonic vibrators is cooled on the object contact surface, and the fact that the object is directly affected by the heat generation of the ultrasonic vibrators is unchanged.

Further, Japanese Utility Model Application Publication JP-U-57-88074 discloses an ultrasonic probe provided, outside of ultrasonic vibrators, with a thermoelectric cooling element in contact with the object, and the thermoelectric cooling element is temperature-controllable for heating or cooling the object by changing the direction of a current flow.

However, as shown in FIG. 1 of JP-U-57-88074, the cooling medium is provided apart from the space where the ultrasonic vibrators are provided, and therefore, only the periphery of the ultrasonic vibrators is cooled on the object contact surface, and the fact that the object is directly affected by the heat generation of the ultrasonic vibrators is unchanged.

Japanese Patent Application Publication JP-P2003-38485A discloses an ultrasonic diagnostic apparatus including an ultrasonic probe provided with ultrasonic vibrators for transmitting and receiving ultrasonic waves, and a channel, through which a medium for transferring heat from the ultrasonic vibrators flows, is formed in the ultrasonic probe, and a circulation mechanism for circulating the medium is connected to the channel.

However, in JP-P2003-38485A, a water bag to be filled with water as the cooling medium is disposed at the living body side of the probe (i.e., before the ultrasonic vibrators), and thereby, the distance between the ultrasonic vibrators and the object becomes longer and causes the attenuation of ultrasonic waves to be transmitted and received by the ultrasonic vibrators.

### SUMMARY OF THE INVENTION

Accordingly, in view of the above-mentioned problems, a purpose of the present invention is, in an ultrasonic probe or an ultrasonic endoscope to be used in an ultrasonic diagnostic apparatus for medical use, to suppress the rise of the temperature of the ultrasonic probe or the ultrasonic endoscope due to heat generated from ultrasonic transducers without causing attenuation of ultrasonic waves transmitted or received by the ultrasonic transducers.

In order to accomplish the purpose, an ultrasonic probe according to one aspect of the present invention includes: an ultrasonic transducer array in which plural ultrasonic transducers are arranged with gaps in between; a casing for accommodating at least the ultrasonic transducer array; and channels for circulation of a liquid heat transfer material in the gaps between the plural ultrasonic transducers.

Further, an ultrasonic endoscope according to one aspect of the present invention includes: an ultrasonic transducer array provided in an insertion part formed of a material having flexibility to be inserted into a body cavity of an object to be inspected, in which plural ultrasonic transducers are arranged with gaps in between; and channels for circulation of a liquid heat transfer material in the gaps between the plural ultrasonic transducers.

Furthermore, an ultrasonic diagnostic apparatus according to one aspect of the present invention includes: the above-mentioned ultrasonic probe or ultrasonic endoscope; drive signal supply means for supplying drive signals to the plural ultrasonic transducers, respectively; signal processing means for generating image data representing an ultrasonic image by processing reception signals outputted from the plural ultrasonic transducers, respectively; and heat transfer material circulating means connected to the channels of the ultrasonic probe or ultrasonic endoscope, for collecting the heat transfer material from the ultrasonic probe or ultrasonic endoscope, cooling the collected heat transfer material, and supplying the cooled heat transfer material to the ultrasonic probe or ultrasonic endoscope.

According to the present invention, since the liquid heat transfer material is flown through the gaps between the plural ultrasonic transducers such that the respective ultrasonic transducers can be directly cooled, the cooling efficiency can be improved. Further, since all of the ultrasonic transducers can be cooled to nearly equal temperatures regardless of arrangement of the ultrasonic transducers, the temperature distribution in the ultrasonic transducer array can be averaged.

### BRIEF DESCRIPTION OF THE DRAWINGS

FIG. 1 is a perspective view showing an exterior appearance and part of an interior of an ultrasonic probe according to the first embodiment of the present invention;

FIG. 2 shows a configuration of an ultrasonic diagnostic apparatus main body to which the ultrasonic probe according to any one of the first to fifth embodiments of the present invention is connected;

FIG. 3 shows a state of the interior of the ultrasonic probe shown in FIG. 1;

FIG. 4 is a partially sectional perspective view showing a single-layer ultrasonic transducer;

FIG. 5 shows a state of an interior of an ultrasonic probe according to the second embodiment of the present invention;

FIG. 6 shows an interior of an ultrasonic probe according to the third embodiment of the present invention;

FIG. 7 is a partially sectional perspective view showing a multilayered ultrasonic transducer;

FIG. 8 is a sectional view showing an ultrasonic probe according to the fifth embodiment of the present invention;

FIG. 9 is a diagram for explanation of a modified example of the ultrasonic diagnostic apparatus main body to which the ultrasonic probe according to the first to fifth embodiments of the present invention is connected;

FIG. 10 is a sectional view showing an ultrasonic probe according to the sixth embodiment of the present invention;

FIG. 11 shows a configuration of an ultrasonic diagnostic apparatus main body to which the ultrasonic probe shown in FIG. 10 is connected;

FIG. 12 is a plan view showing an interior of an ultrasonic probe according to the seventh embodiment of the present invention;

FIG. 13 is a schematic diagram showing a configuration of an ultrasonic endoscope according to one embodiment of the present invention; and

FIG. 14 is an enlarged schematic diagram showing the leading end of an insertion part shown in FIG. 13.

### DESCRIPTION OF THE PREFERRED EMBODIMENTS

Hereinafter, embodiments of the present invention will be explained in detail with reference to the drawings. The same reference numbers will be assigned to the same component elements and the description thereof will be omitted.

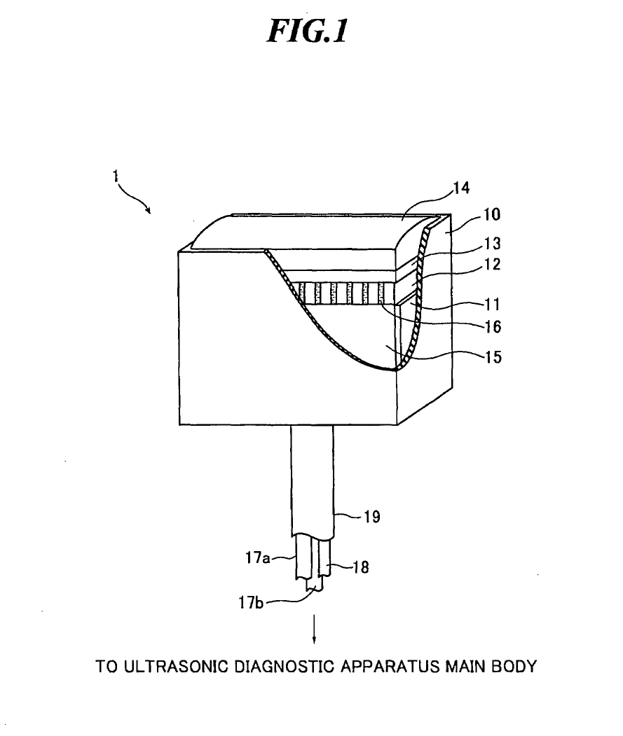

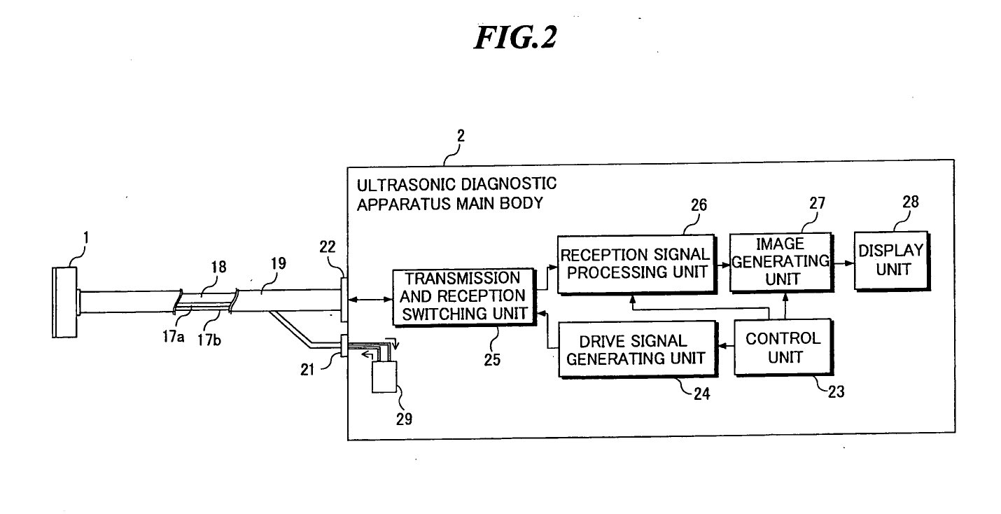

FIG. 1 is a perspective view showing an exterior appearance and part of an interior of an ultrasonic probe according to the first embodiment of the present invention. The ultrasonic probe is used in contact with an object to be inspected when extracavitary scanning is performed. Further, FIG. 2 shows a configuration of an ultrasonic diagnostic apparatus main body to which the ultrasonic probe according to any one of the first to fifth embodiments of the present invention is connected.

As shown in FIG. 1, a head part 1 of the ultrasonic probe includes a casing 10, and a backing layer 11, an ultrasonic transducer array 12 having plural ultrasonic transducers, an acoustic matching layer 13, an acoustic lens 14, and flexible printed circuits (FPCs) 15, which are accommodated within the casing 10, and a liquid heat transfer material (heat transfer medium) 16 flowing through gaps between the plural ultrasonic transducers as channels. Further, two circulation tubes 17a and 17b as channels for circulating the heat transfer material 16, an electric cable 18, and a cable cover 19 for protecting them are connected to the casing 10.

As shown in FIG. 2, the circulation tubes 17a and 17b extending from the head part 1 are connected to an ultrasonic diagnostic apparatus main body 2 via a circulation medium connecter 21, and provided for circulation of the heat transfer material 16 between the head part 1 and the ultrasonic diagnostic apparatus main body 2. Further, the head part 1 is electrically connected to the ultrasonic diagnostic apparatus main body 2 via the electric cable 18 and an electric connector 22. The electric cable 18 transmits drive signals generated by the ultrasonic diagnostic apparatus main body 2 to the ultrasonic transducers and transmits reception signals outputted from the respective ultrasonic transducers to the ultrasonic diagnostic apparatus main body 2.

The ultrasonic diagnostic apparatus main body 2 includes a control unit 23 for controlling the operation of the entire system including the ultrasonic probe and the ultrasonic diagnostic apparatus main body 2, a drive signal generating unit 24, a transmission and reception switching unit 25, a reception signal processing unit 26, an image generating unit 27, a display unit 28, and a cooler 29 with a circulation pump. The drive signal generating unit 24 generates drive signals to be used for respectively driving the plural ultrasonic transducers. The transmission and reception switching unit 25 switches output of drive signals to the ultrasonic probe and input of reception signals from the ultrasonic probe. The reception signal processing unit 26 performs predetermined signal processing of amplification, phasing addition, detection, etc. on the reception signals outputted from the respective ultrasonic transducers. The image generating unit 27 generates image data representing an ultrasonic image based on the reception signals on which the predetermined signal processing has been performed. The display unit 28 displays the ultrasonic image based on thus generated image data.

The cooler 29 with the circulation pump cools the heat transfer material 16, and supplies the cooled heat transfer material to the head part 1 via the circulation tube 17a and collects the heat transfer material 16 that has passed through the channels within the head part 1 to the cooler 29 via the circulation tube 17b.

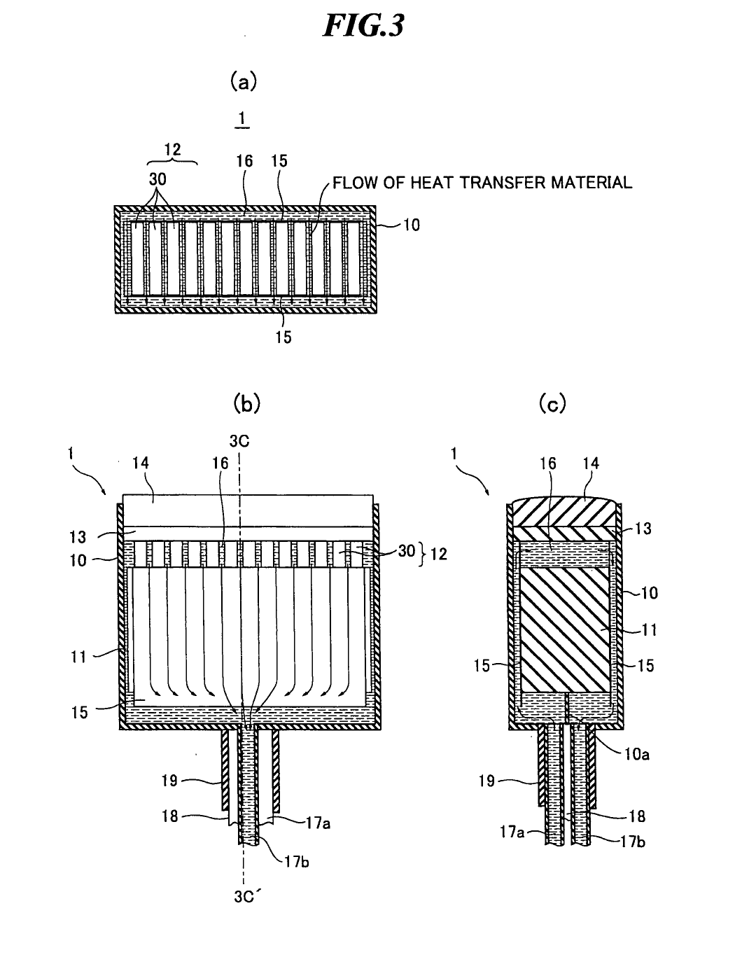

FIG. 3(a) is a front view showing an interior of the head part 1 of the ultrasonic probe shown in FIG. 1, and FIG. 3(b) is a plan view showing an interior of the ultrasonic probe shown in FIG. 1. Further, FIG. 3(c) is a sectional view along the dashed-dotted line 3C-3C′ shown in FIG. 3(b). Here, the arrows shown in FIG. 3(a) to (c) indicate the flow directions of the heat transfer material 16. In FIG. 3(a), the acoustic matching layer 13 and the acoustic lens 14 shown in FIG. 1 are omitted.

As shown in FIG. 3(a), the ultrasonic transducer array 12 includes plural ultrasonic transducers 30 arranged in a one-dimensional form.

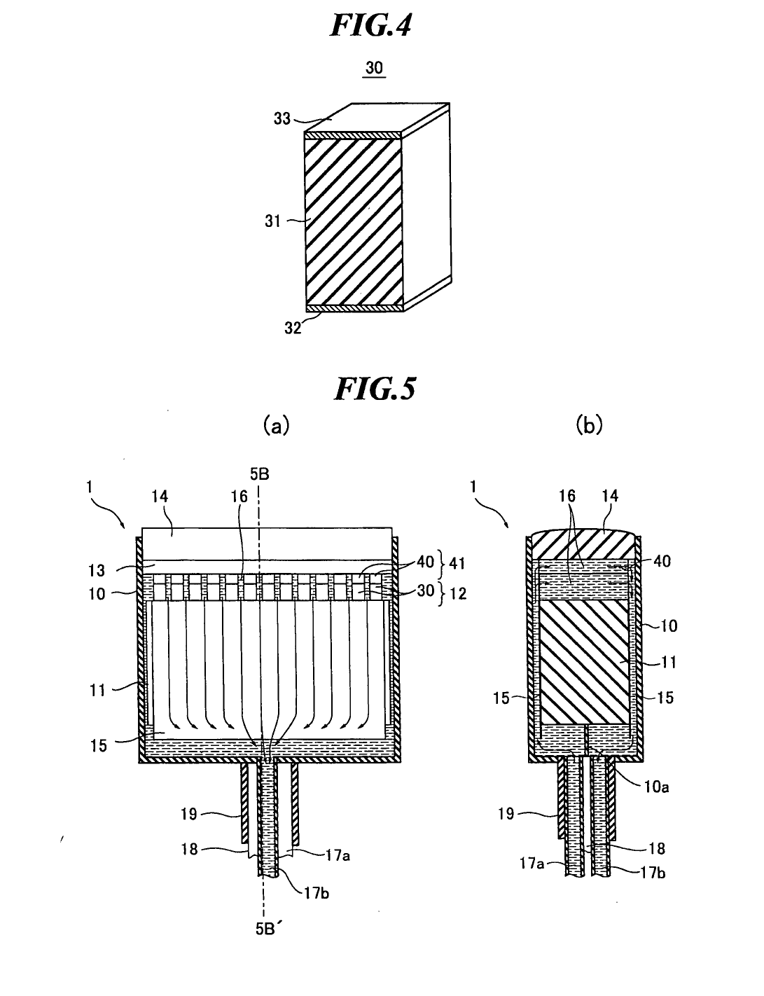

Here, referring to FIG. 4, each ultrasonic transducer 30 is a vibrator including a piezoelectric material 31 of PZT (Pb (lead) zirconate titanate) and electrodes 32 and 33 formed on two opposing surfaces of the piezoelectric material 31. One of the electrodes 32 and 33 is used for common connection among the plural ultrasonic transducers.

The ultrasonic transducers 30 generate ultrasonic waves based on the drive signals supplied from the ultrasonic diagnostic apparatus main body. Further, the ultrasonic transducers 30 receive ultrasonic echoes propagating from the object and generate electric signals. The electric signals are outputted to the ultrasonic diagnostic apparatus main body and processed as reception signals of the ultrasonic echoes.

As shown in FIG. 3(a), the plural ultrasonic transducers 30 are arranged at predetermined intervals (e.g., about 0.1 mm to 1 mm) on the backing layer 11. As a result, gaps formed between the plural ultrasonic transducers constitute part of the channels for the heat transfer material 16.

Referring to FIG. 3(b), the backing layer 11 is formed of a material having large acoustic attenuation such as an epoxy resin including ferrite powder, metal powder, or PZT powder, or rubber including ferrite powder, on the opposite side to ultrasonic transmission/reception surfaces of the ultrasonic transducers 30, which surfaces transmit ultrasonic waves toward the object and receive ultrasonic waves propagating from the object. The backing layer 11 promotes attenuation of unwanted ultrasonic waves generated from the ultrasonic transducer array 12.

The acoustic matching layer 13 is formed of, for example, Pyrex (registered trademark) glass or an epoxy resin including metal powder, which easily propagates ultrasonic waves, on the ultrasonic transmission/reception surfaces of the ultrasonic transducers 30. The acoustic matching layer 13 resolves the mismatch of acoustic impedances between the object as a living body and the ultrasonic transducers. Thereby, the ultrasonic waves transmitted from the ultrasonic transducers efficiently propagate within the object. Although the single-layer acoustic matching layer 13 has been shown in FIGS. 1 and 3, plural acoustic matching layers may be provided according to need.

The acoustic lens 14 is formed of, for example, silicon rubber, and focuses an ultrasonic beam, which is transmitted from the ultrasonic transducer array 12 and propagates through the acoustic matching layer 13, at a predetermined depth within the object.

Wirings connected to the respective transducers 30 are formed on two FPCs 15. One end of each wiring is connected to the electrode of the ultrasonic transducer 30 and the other end thereof is connected to the electric cable 18. In FIG. 3(b) and (c), the connection configuration between the FPCs 15 and the electric cable 18 is omitted for easy understanding of the flow condition of the heat transfer material 16.

The heat transfer material 16 is a liquid for passing through the channels within the casing 10 to absorb the heat generated from the ultrasonic transducers 30. As the heat transfer material 16, a material having good heat transference and electric isolation is used. The electric isolation is necessary because the heat transfer material 16 circulates within the casing 10 in direct contact with the ultrasonic transducers 30. As a material that satisfies the condition, liquid paraffin, silicone oil, water, alcohol, mixture of water and alcohol, and fluorinated inert liquid are cited. Among them, liquid paraffin, silicone oil, and fluorinated inert liquid (e.g., FLUORINERT (registered trademark) manufactured by Sumitomo 3M) are preferable on the point that they have high fluidity, high electric insulation, and advantageous safety. In the embodiment, the liquid paraffin is used.

As shown in FIG. 3(c), the heat transfer material 16 introduced from the ultrasonic diagnostic apparatus main body via the circulation tube 17a into the casing 10 sequentially passes the outer side of one FPC 15, the gaps between the adjacent ultrasonic transducers 30, and the outer side of the other FPC 15, led out from the casing 10, and collected in the ultrasonic diagnostic apparatus main body via the circulation tube 17b. A partition 10a may be provided within the casing 10 for forming a flow in one direction.

As described above, in the embodiment, the heat transfer material 16 cooled in the ultrasonic diagnostic apparatus main body 2 is flown through the gaps between the ultrasonic transducers 30, and thereby, the heat can be directly absorbed from the respective ultrasonic transducers 30. Therefore, the plural ultrasonic transducers 30 can be uniformly cooled, and the central part of the ultrasonic transducer array 12, in which heat especially tends to stay, can be sufficiently and evenly cooled. Thus, the temperature distribution in the plural ultrasonic transducer arrays is averaged and the influence by the temperature on the ultrasonic transmission and reception operation such as sensitivity variations or the like can be suppressed. Further, the heat transfer material 16 also passes the periphery of the FPCs 15, and thereby, the temperature rise of the circuits can be suppressed and the operation of the ultrasonic probe can be stabilized.

Next, an ultrasonic probe according to the second embodiment of the present invention will be explained with reference to FIG. 5. FIG. 5(a) is a plan view showing an interior of the ultrasonic probe according to the embodiment, and FIG. 5(b) is a sectional view along the dashed-dotted line 5B-5B′ shown in FIG. 5(a).

As shown in FIG. 5(a), the ultrasonic probe according to the embodiment has an acoustic matching layer 41 in place to the acoustic matching layer 13 shown in FIG. 3. The acoustic matching layer 41 includes plural acoustic matching members 40 placed on the plural ultrasonic transducers 30, respectively. The rest of the configuration is the same as that in the first embodiment.

In the case where the acoustic matching members are separately placed in this manner, the propagation directions of ultrasonic waves in the respective acoustic matching members 40 are narrowed down to some degree, and therefore, the propagation efficiency of ultrasonic waves at a boundary (e.g., a boundary between the acoustic matching member 40 and the acoustic lens 14) can be improved. Further, as shown in FIG. 5(b), the heat transfer material 16 circulates in a broader area within the casing 10, and therefore, the temperature rise of the ultrasonic transducer array 12 can be suppressed more efficiently.

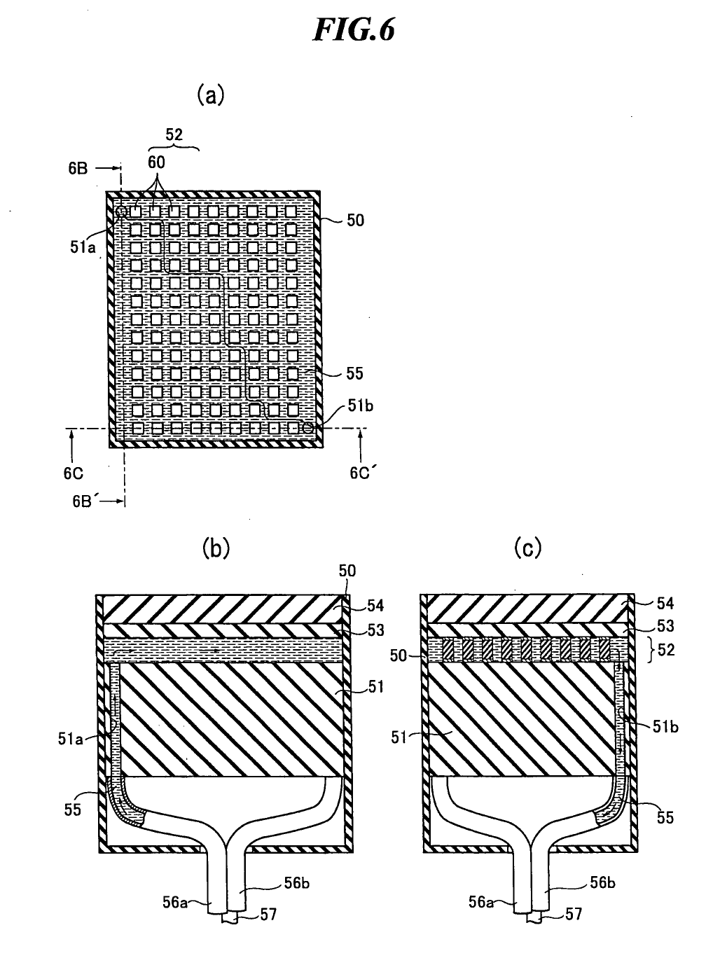

Next, an ultrasonic probe according to the third embodiment of the present invention will be explained with reference to FIG. 6.

The ultrasonic probe according to the embodiment has an ultrasonic transducer array in which plural ultrasonic transducers are two-dimensionally arranged, and accordingly, the channel configuration formed within the head part is different from that in the first embodiment. The ultrasonic diagnostic apparatus to which the ultrasonic probe according to the embodiment is connected and the connection configuration between the ultrasonic probe and the ultrasonic diagnostic apparatus main body are the same as those have been explained with reference to FIG. 2.

FIG. 6(a) is a front view showing an interior of a head part of the ultrasonic probe according to the embodiment. Further, FIG. 6(b) is a sectional view along the dashed-dotted line 6B-6B′ shown in FIG. 6(a), and FIG. 6(c) is a sectional view along the dashed-dotted line 6C-6C′ shown in FIG. 6(a). In FIG. 6(a), an acoustic matching layer 53 and an acoustic lens 54 shown in FIG. 6(b) are omitted.

As shown in FIG. 6(a) to (c), the head part of the ultrasonic probe according to the embodiment includes a casing 50, and a backing layer 51, an ultrasonic transducer array 52, the acoustic matching layer 53, and the acoustic lens 54, which are accommodated within the casing 50, and a liquid heat transfer material 55 flowing through gaps in the ultrasonic transducer array 52. Further, the head part is connected to the ultrasonic diagnostic apparatus main body via circulation tubes 56a and 56b and an electric cable 57. The materials forming the backing layer 51, the acoustic matching layer 53, the acoustic lens 54, and the heat transfer material 55 and functions thereof are the same as those in the first embodiment.

In the ultrasonic transducer array 52, plural ultrasonic transducers 60 are arranged in a two-dimensional matrix form. The gaps between these ultrasonic transducers 60 form two-dimensional channels for the heat transfer material 55. Further, each ultrasonic transducer 60 has a structure including a piezoelectric material layer and electrode layers formed both sides thereof like that shown in FIG. 4.

As shown in FIG. 6(a), two holes 51a and 51b are formed at two corners of the backing layer 51. As shown in FIG. 6(b) and (c), the circulation tube 56a is connected to the hole 51a and the circulation tube 56b is connected to the hole 51b. The heat transfer material 55 supplied into the casing 50 via the circulation tube 56a passes through the hole 51a and is introduced into the ultrasonic transducer array 52, and two-dimensionally spreads into the gaps between the ultrasonic transducers 60. Then, the heat transfer material 55 flows into the hole 51b diagonally opposite in the front view to the hole 51a in the backing layer, and is collected by the circulation tube 56b.

Here, in the two-dimensional ultrasonic transducer array, the heat generated from the ultrasonic transducers located inner side is especially hard to disperse, and the heat especially tends to stay around the center. However, in the embodiment, the heat transfer material is flown through the gaps between the ultrasonic transducers, and thereby, even the ultrasonic transducers around the center can be sufficiently cooled. Therefore, the production of a temperature gradient can be suppressed in the ultrasonic transducer array, and thus, the influence due to temperature such as variations in detection sensitivity of ultrasonic waves can be suppressed.

In the embodiment, the holes 51a and 51b are formed in two locations at two corners of the backing layer 51, however, the holes may be formed in other locations as long as the heat transfer material can be evenly circulated in the gaps of the ultrasonic transducer array 52 and the heat transfer material can be smoothly led out. Further, more than two holes may be provided.

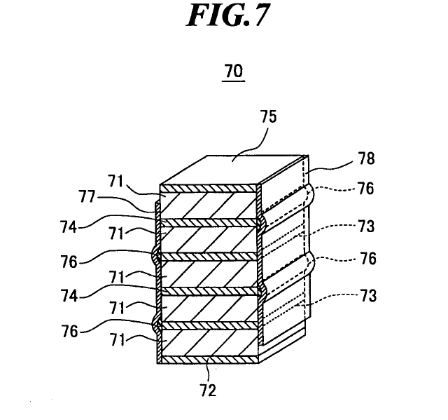

Next, an ultrasonic transducer array according to the fourth embodiment of the present invention will be explained with reference to FIGS. 6 and 7.

In the embodiment, a multi layered ultrasonic transducer 70 shown in FIG. 7 is applied to the ultrasonic probe shown in FIG. 6 in place of the single-layer ultrasonic transducer (see FIG. 4).

The multilayered ultrasonic transducer 70 shown in FIG. 7 includes plural piezoelectric material layers 71 formed of PZT or the like, a lower electrode layer 72, internal electrode layers 73 and 74, an upper electrode layer 75, insulating films 76, and side electrodes 77 and 78.

The lower electrode layer 72 is connected to the side electrode 77 on the left side in the drawing and insulated from the side electrode 78 on the right side in the drawing. Further, the internal electrode layers 73 and 74 are alternately inserted between the plural piezoelectric material layers 71. The internal electrode layers 73 are connected to the side electrode 78 and insulated from the side electrode 77 by the insulating films 76. On the other hand, the internal electrode layers 74 are connected to the side electrode 77 and insulated from the side electrode 78 by the insulating films 76. Furthermore, the upper electrode layer 75 is connected to the side electrode 78 and insulated from the side electrode 77. The plural electrodes of the ultrasonic transducer are thus formed, and thereby, five sets of electrodes for applying electric fields to the five layers of piezoelectric material layers 71 are connected in parallel. The number of the piezoelectric material layers is not limited to five as shown in FIG. 7, but two to four or six or more layers may be provided.

In the multilayered ultrasonic transducer (here, also referred to as “element”), areas of facing electrodes are larger than that in the single-layer element, and the electric impedance is lower. Therefore, the multilayered element operates more efficiently for an applied voltage than the single-layer element. Specifically, given that the number of the piezoelectric material layers is N (N=5 in FIG. 7), the number of the piezoelectric material layers is N times the number of the single-layer element and the thickness of each piezoelectric material layer is 1N times the thickness of the single-layer element, and therefore, the electric impedance of the element is 1N² times the electric impedance of the single-layer element. Accordingly, the electric impedance of the element can be adjusted by increasing and decreasing the number of stacked layers of the piezoelectric material layers, and thus, the matching with the drive circuit and/or the preamplifier can be easily achieved and the sensitivity can be improved.

On the other hand, the capacitance increases due to stacked form of the element, and the amount of heat generated from each element increases. However, as shown in FIG. 6, the heat transfer material 55 is flown between the plural elements, and thereby, the respective elements can be directly and efficiently cooled. Therefore, even when the amounts of heat generated from the multilayered elements increase, the temperature rise of the ultrasonic probe can be suppressed.

Such multilayered ultrasonic transducers may be applied to the one-dimensional ultrasonic transducer array shown in FIG. 3.

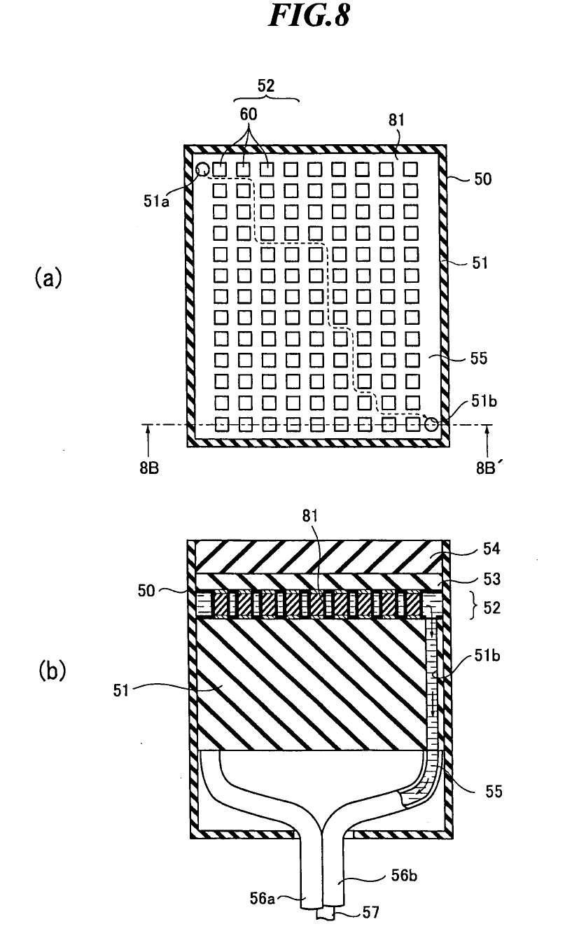

Next, an ultrasonic probe according to the fifth embodiment of the present invention will be explained with reference to FIG. 8. FIG. 8(a) is a front view showing an interior of a head part of the ultrasonic probe according to the embodiment, and FIG. 8(b) is a sectional view along the dashed-dotted line 8B-8B′ shown in FIG. 8(a). In FIG.8(a), the acoustic matching layer 53 and the acoustic lens 54 shown in FIG. 8(b) are omitted.

As shown in FIG. 8(b), in the ultrasonic probe according to the embodiment, insulating films 81 formed of a resin material are formed to cover the walls in the channels for the heat transfer material 55 filling the gaps between the ultrasonic transducers 60 in the ultrasonic transducer array 52 shown in FIG. 6. The rest of the configuration is the same as that shown in FIG. 6.

In the previously explained first to fourth embodiments, the heat transfer material is flown in the periphery of the ultrasonic transducers, and the heat transfer material directly contact the electrode parts and element joint parts. Accordingly, there may be concerns about operation reliability when water, mixture of water and alcohol, or the like is used, while not so much problematic when a liquid with high insulation such as liquid paraffin is used.

On this account, in the embodiment, the electric reliability is improved by covering the channels for the heat transfer material with the insulating resin material. The insulating films 81 are formed to cover at least around the ultrasonic transducers 60 in the channels, and desirably, they are formed on the floor surface (the upper surface of the backing layer 51) and the ceiling surface (the lower surface of the acoustic matching layer 53) as well. The resin material is used not to obstruct the expanding and contracting motion of the ultrasonic transducers due to the piezoelectric effect. As the resin material, specifically, epoxy resin, urethane resin, silicone resin, polyimide resin, acrylic resin, or the like is used.

The insulating films 81 are formed in the following manner, for example. That is, first, plural ultrasonic transducers 60 are arranged on the backing layer 51 and further the acoustic matching layer 53 is disposed thereon, and thus, the channels for the heat transfer material 55 are formed. Subsequently, the liquid insulating resin material is poured into the channels and the excessive insulating resin material is removed before cured. Then, the insulating resin material attached to the walls of the channels is cured.

Alternatively, as another method of forming the insulating films 81, the channels of the heat transfer material 55 are formed by the backing layer 51, the plural ultrasonic transducers 60, and the acoustic matching layer 53, and the insulating resin material is poured into the channels and cured. Then, the insulating resin material is drilled, and thereby, the two-dimensional channels covered by the insulating films are formed.

In the embodiment, the ultrasonic transducers 60 are indirectly cooled by the heat transfer material 55 via the insulating films 81. However, the respective ultrasonic transducers 60 can be efficiently cooled by selecting an insulating resin material with relatively high heat conductivity or reducing the thickness of the insulating films 81.

Further, insulating films may be formed of a resin material in the one-dimensional ultrasonic transducer array shown in FIG. 3.

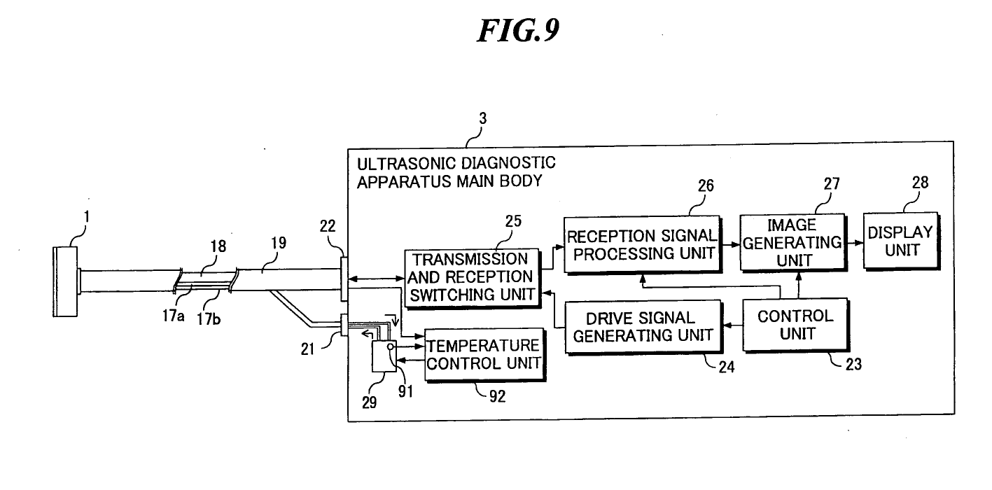

Next, a modified example of the ultrasonic diagnostic apparatus main body to which the ultrasonic probe according to any one of the first to fifth embodiments of the present invention is connected will be explained with reference to FIG. 9.

The ultrasonic diagnostic apparatus main body 3 shown in FIG. 9 further has a temperature sensor 91 and a temperature control unit 92 compared to the ultrasonic diagnostic apparatus main body 2 shown in FIG. 2. The rest of the configuration is the same as that shown in FIG. 2.

The temperature sensor 91 specifically includes a thermistor, thermocouple, or the like. The temperature sensor 91 is attached to the cooler 29 with the circulation pump, and senses the temperature of the heat transfer material collected from the head part 1 via the circulation tube 17b or 56b. The temperature control unit 92 obtains a value on the temperature of the heat transfer material based on a sensing result of the temperature sensor 91, and controls the operation of the cooler 29 with the circulation pump based on the obtained value. For example, when the obtained value on the temperature of the heat transfer material exceeds a predetermined value, the temperature control unit 92 lowers the preset temperature of the cooler 29 or increases the pressure of the circulation pump for increasing the flow rate of the heat transfer material within the head part 1. Alternatively, the cooler 29 with the circulation pump may be operated only when the obtained value on the temperature of the heat transfer material exceeds the predetermined value.

According to the embodiment, since the operation of the cooler 29 with circulation pump is feedback-controlled based on the temperature of the heat transfer material, the temperature of the heat transfer material can be easily kept in a certain range and the operation cost of the cooler 29 with circulation pump can be reduced.

As a modified example of the ultrasonic diagnostic apparatus main body shown in FIG. 9, a calculating unit for calculating the temperature based on the sensing result of the temperature sensor 91 may be provided in place of the temperature control unit 92, and the control unit 23 may control the cooler 29 with the circulation pump based on a calculation result thereof.

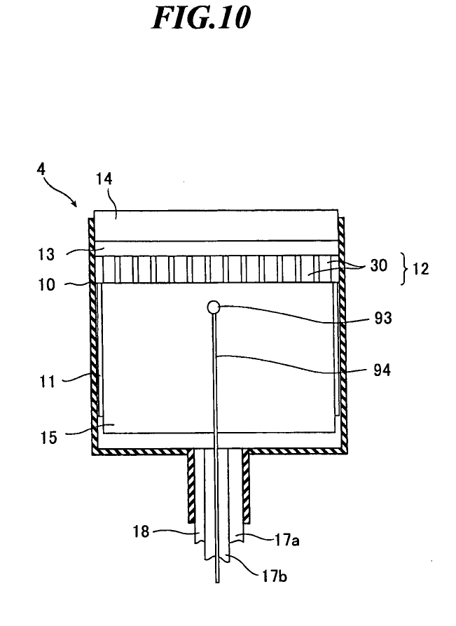

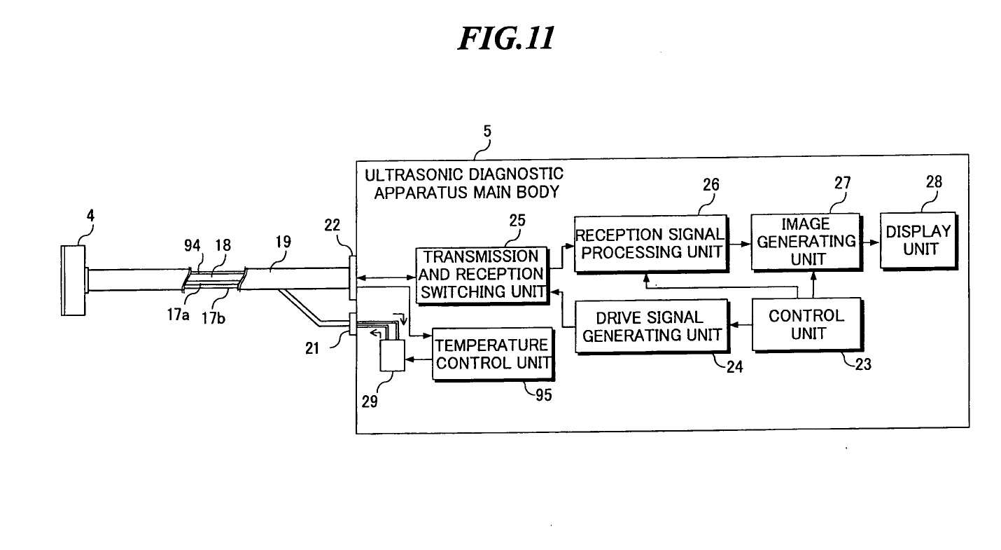

Next, an ultrasonic probe according to the sixth embodiment of the present invention will be explained with reference to FIGS. 10 and 11. FIG. 10 is a plan view showing an interior of the ultrasonic probe according to the embodiment, and FIG. 11 shows a configuration of an ultrasonic diagnostic apparatus main body to which the ultrasonic probe is connected.

As shown in FIG. 10, the ultrasonic probe according to the embodiment further includes a temperature sensor 93 for sensing the temperature within the head part 4 compared to the ultrasonic probe shown in FIG. 3. The rest of the configuration is the same as that shown in FIG. 3.

The temperature sensor 93 specifically includes a thermistor, thermocouple, or the like, and is attached to the surface of the FPC 15. Alternatively, the temperature sensor 93 may be embedded in the backing layer 11 or disposed on the surface of the backing layer 11 (i.e., in the channel of the heat transfer material). In either case, the temperature sensor 93 is desirably located as close as possible to the ultrasonic transducer 30. Further, the temperature sensor 93 is electrically connected to an ultrasonic diagnostic apparatus main body 5 (FIG. 11) by a lead wire 94.

As shown in FIG. 11, the ultrasonic diagnostic apparatus main body 5 to be used in the embodiment has a temperature control unit 95. The rest of the configuration of the ultrasonic diagnostic apparatus main body 5 is the same as that has been described with reference to FIG. 2.

The temperature control unit 95 obtains a value on the temperature of the heat transfer material based on a sensing result of the temperature sensor 93 received via the lead wire 94, and controls the operation of the cooler 29 with the circulation pump based on the obtained value such that the temperature of the head part 4 falls within a desired range. For example, when the obtained value on the temperature within the head part exceeds a predetermined value, the temperature control unit 95 lowers the preset temperature of the cooler or increases the pressure of the circulation pump. Alternatively, the cooler 29 with the circulation pump may be operated only when the obtained value on the temperature within the head part exceeds the predetermined value.

According to the embodiment, since the operation of the cooler 29 with circulation pump is feedback-controlled based on the temperature within the head part 4 of the ultrasonic probe, the temperature within the head part 4 can be controlled more accurately and the operation cost of the cooler 29 with circulation pump can be reduced.

Also in the embodiment, a calculating unit for calculating a value on the temperature within the head part 4 based on the sensing result of the temperature sensor 93 may be provided in place of the temperature control unit 95, and the control unit 23 may control the cooler 29 with the circulation pump based on a calculation result thereof.

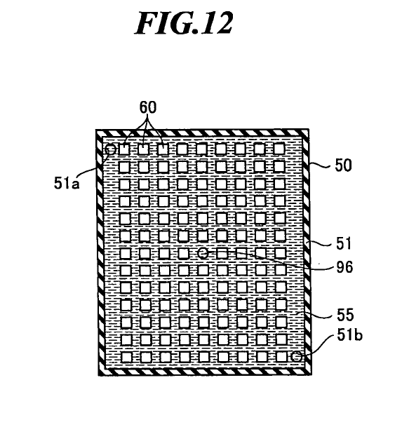

Next, an ultrasonic probe according to the seventh embodiment of the present invention will be explained with reference to FIG. 12. An ultrasonic diagnostic apparatus main body to which the ultrasonic probe according to the embodiment is connected is the same as that shown in FIG. 11.

As shown in FIG. 12, in the ultrasonic probe according to the embodiment, a temperature sensor 96 is provided in place of part of the ultrasonic transducers 60 in a two-dimensional ultrasonic transducer array in which the plural ultrasonic transducers 60 are arranged. The rest of the configuration of the ultrasonic probe is the same as that shown in FIG. 6.

In FIG. 12, the temperature sensor 96 is located near the central part of the ultrasonic transducer array. This is because the part around the center is a region in which heat tends to stay and the temperature is most likely to rise. However, the temperature sensor 96 may be located in other positions, or plural temperature sensors 96 may be provided in plural regions, respectively. The sensing result of the temperature sensor 96 is used for controlling the cooler 29 with the circulation pump in the ultrasonic diagnostic apparatus main body shown in FIG. 11.

In the above explanation, in the cooling mechanism incorporated into the ultrasonic diagnostic apparatus main body, the heat transfer material circulated within the ultrasonic probe is cooled and fed with pressure to the head part of the ultrasonic probe, however, an independent cooling mechanism (cooler with circulation pump) may be provided separately from the ultrasonic diagnostic apparatus main body. In this case, only the electric cable 18 of the ultrasonic probe shown in FIG. 2 may be connected to the ultrasonic diagnostic apparatus main body, and thereby, the ultrasonic probe with cooling mechanism can be used in a conventional ultrasonic diagnostic apparatus.

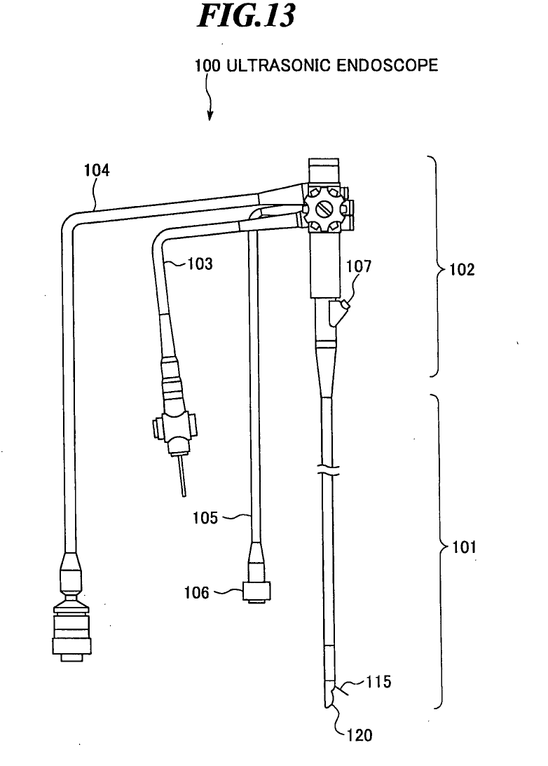

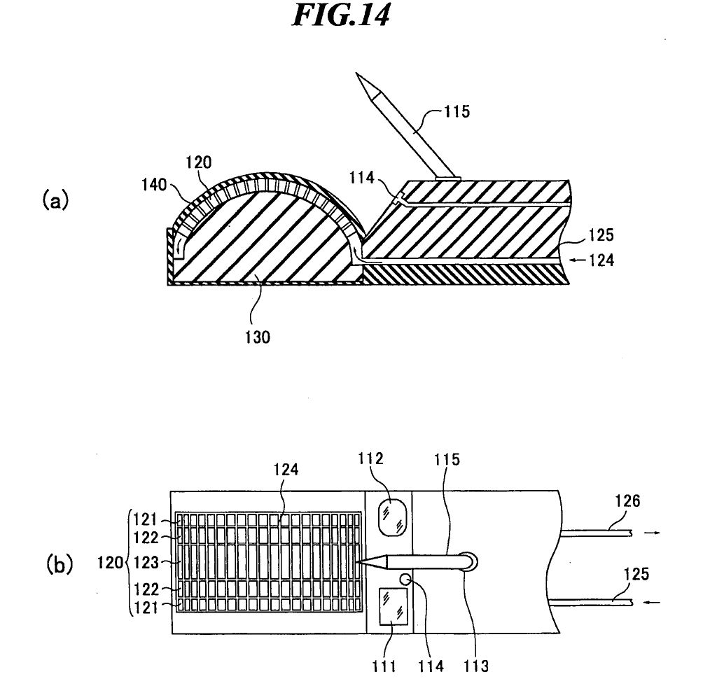

Next, an ultrasonic endoscope according to one embodiment of the present invention will be explained with reference to FIGS. 13 and 14. Here, the ultrasonic endoscope is an instrument having an ultrasonic probe for intracavitary provided at the leading end of an insertion part of an endoscopic examination device for optical observation of the intracavitary of the object. The ultrasonic endoscope is connected to the ultrasonic diagnostic apparatus main body as shown in FIG. 2, 9 or 11 to configure an ultrasonic diagnostic apparatus.

FIG. 13 is a schematic diagram showing an appearance of the ultrasonic endoscope. As shown in FIG. 13, the ultrasonic endoscope 100 includes an insertion part 101, an operation part 102, a connecting cord 103, a universal cord 104, a circulation medium cable 105, and a circulation medium connector 106.

The insertion part 101 of the ultrasonic endoscope 100 is an elongated tube formed of a material having flexibility to be inserted into the body of the object. The operation part 102 is provided at the base end of the insertion part 101, connected to the ultrasonic diagnostic apparatus main body via the connecting cord 103, and connected to a light source unit via the universal cord 104.

FIG. 14 is an enlarged schematic diagram showing the leading end of the insertion part 101 shown in FIG. 13. FIG. 14(a) shows the leading end of the insertion part 101 seen from side, and FIG. 14(b) shows the leading end seen from above.

As shown in FIG. 14(a) and (b), at the leading end of the insertion part 101, an observation window 111, an illumination window 112, a treatment tool passage opening 113, a nozzle hole 114, and an ultrasonic transducer array 120 are provided. A punctuation needle 115 is provided in the treatment tool passage opening 113.

An objective lens is fit in the observation window 111, and an input end of an image guide or a solid-state image sensor such as a CCD camera is provided in the imaging position of the objective lens. These configure an observation optical system. Further, an illumination lens for outputting illumination light to be supplied from the light source unit via a light guide is fit in the illumination window 112. These configure an illumination optical system.

The treatment tool passage opening 113 is a hole for leading out a treatment tool inserted from a treatment tool insertion opening 107 (FIG. 13) provided in the operation part 102. Various treatments are performed within a body cavity of the object by projecting the treatment tool such as the punctuation needle 115 or forceps from the hole and operating it with the operation part 102. Furthermore, the nozzle hole 114 is provided for injecting a liquid (water or the like) for cleaning the observation window 111 and the illumination window 112.

As shown in FIG. 14(a), a backing layer 130 is provided on the back of the ultrasonic transducer array 120, and an acoustic matching layer 140 is provided on the front of the ultrasonic transducer array 120. An acoustic lens is provided on the acoustic matching layer 140 according to need.

In FIG. 14(b), the acoustic matching layer 140 is omitted to show the ultrasonic transducer array 120. As shown in FIG. 14(b), the ultrasonic transducer array 120 is a convex-type multirow array and includes plural ultrasonic transducers 121-123 arranged in five rows on a curved surface. The gaps between the ultrasonic transducers 121-123 are channels for a heat transfer material 124. Further, a circulation tube 125 for supplying the heat transfer material and a circulation tube 126 for collecting the heat transfer material are connected to the channels. The circulation tubes 125 and 126 are accommodated in the circulation medium cable 105 (see FIG. 13) and connected to a cooling unit provided inside or outside of the ultrasonic diagnostic apparatus main body. The heat transfer material circulates between the channels of the ultrasonic transducer array 120 and the cooling unit via the circulation tubes 125 and 126.

Here, in FIG. 14, the convex-type multirow array is shown as the ultrasonic transducer array 120, however, a radial-type multirow array in which plural ultrasonic transducers are arranged on a cylindrical surface or an ultrasonic transducer array in which plural ultrasonic transducers are arranged on a spherical surface may be used for the ultrasonic endoscope.

As described above, since the heat transfer material is flown through the channels of the ultrasonic transducer array 120, the ultrasonic transducers 121-123 can be directly cooled. Thereby, the temperature rise of the ultrasonic endoscope is suppressed and the safety in ultrasonic endoscopic examination can be improved.

Also in the ultrasonic endoscopic shown in FIG. 14, the temperature sensor for measuring the temperature in the leading end of the insertion part 101 in the vicinity of the ultrasonic transducer array may be provided for feedback-control of the cooling unit of the heat transfer material based on the measurement value of the temperature of the heat transfer material. Further, insulating films may be formed of a resin material in the channels of the ultrasonic transducer array shown in FIG. 14 for improvement of insulation of the ultrasonic transducers.

1. An ultrasonic probe comprising:
an ultrasonic transducer array in which plural ultrasonic transducers are arranged with gaps in between;
a casing for accommodating at least said ultrasonic transducer array; and
channels for circulation of a liquid heat transfer material in the gaps between said plural ultrasonic transducers.

2. The ultrasonic probe according to claim 1, further comprising:
a backing layer arranged at an opposite side to an ultrasonic transmission/reception surface of said ultrasonic transducer array.

3. The ultrasonic probe according to claim 1, further comprising:
at least one acoustic matching layer arranged at an ultrasonic transmission/reception surface side of said ultrasonic transducer array.

4. The ultrasonic probe according to claim 1, further comprising:
an acoustic lens directly or indirectly arranged at an ultrasonic transmission/reception surface side of said ultrasonic transducer array.

5. The ultrasonic probe according to claim 1, further comprising:
insulating films formed on surfaces of said plural ultrasonic transducers in the gaps between said plural ultrasonic transducers.

6. The ultrasonic probe according to claim 1, wherein each of said plural ultrasonic transducers has plural piezoelectric material layers and plural internal electrode layers provided between said plural piezoelectric material layers.

7. The ultrasonic probe according to claim 1, wherein said heat transfer material includes one of liquid paraffin, silicone oil, water, alcohol, mixture of water and alcohol, and fluorinated inert liquid.

8. The ultrasonic probe according to claim 1, wherein said channels include:
a lead-in path for leading said heat transfer material into said casing; and
a lead-out path for leading said heat transfer material from said casing.

9. An ultrasonic endoscope comprising:
an ultrasonic transducer array provided in an insertion part formed of a material having flexibility to be inserted into a body cavity of an object to be inspected, in which plural ultrasonic transducers are arranged with gaps in between; and
channels for circulation of a liquid heat transfer material in the gaps between said plural ultrasonic transducers.

10. An ultrasonic diagnostic apparatus comprising:
an ultrasonic probe including an ultrasonic transducer array in which plural ultrasonic transducers are arranged with gaps in between, a casing for accommodating at least said ultrasonic transducer array, and channels for circulation of a liquid heat transfer material in the gaps between said plural ultrasonic transducers;
drive signal supply means for supplying drive signals to said plural ultrasonic transducers, respectively;
signal processing means for generating image data representing an ultrasonic image by processing reception signals outputted from said plural ultrasonic transducers, respectively; and
heat transfer material circulating means connected to the channels of said ultrasonic probe, for collecting the heat transfer material from said ultrasonic probe, cooling the collected heat transfer material, and supplying the cooled heat transfer material to said ultrasonic probe.

11. The ultrasonic diagnostic apparatus according to claim 10, further comprising:
temperature sensing means for sensing a temperature of the heat transfer material collected by said heat transfer material circulating means; and
temperature control means for controlling an operation of said heat transfer material circulating means based on a sensing result of said temperature sensing means.

12. The ultrasonic diagnostic apparatus according to claim 10, wherein:
said ultrasonic probe further includes temperature sensing means for sensing a temperature of said heat transfer material; and
said ultrasonic diagnostic apparatus further comprises temperature control means for controlling an operation of said heat transfer material circulating means based on a sensing result of said temperature sensing means.

13. The ultrasonic diagnostic apparatus according to claim 10, wherein:
said ultrasonic probe further includes temperature sensing means for sensing a temperature within a head part of said ultrasonic probe; and
said ultrasonic diagnostic apparatus further comprises temperature control means for controlling an operation of said heat transfer material circulating means based on a sensing result of said temperature sensing means.

14. An ultrasonic diagnostic apparatus comprising:
an ultrasonic endoscope including an ultrasonic transducer array provided in an insertion part formed of a material having flexibility to be inserted into a body cavity of an object to be inspected, in which plural ultrasonic transducers are arranged with gaps in between, and channels for circulation of a liquid heat transfer material in the gaps between said plural ultrasonic transducers;
drive signal supply means for supplying drive signals to said plural ultrasonic transducers, respectively;
signal processing means for generating image data representing an ultrasonic image by processing reception signals outputted from said plural ultrasonic transducers, respectively; and
heat transfer material circulating means connected to the channels of said ultrasonic endoscope, for collecting the heat transfer material from said ultrasonic endoscope, cooling the collected heat transfer material, and supplying the cooled heat transfer material to said ultrasonic endoscope.

15. The ultrasonic diagnostic apparatus according to claim 14, further comprising:
temperature sensing means for sensing a temperature of the heat transfer material collected by said heat transfer material circulating means; and
temperature control means for controlling an operation of said heat transfer material circulating means based on a sensing result of said temperature sensing means.

16. The ultrasonic diagnostic apparatus according to claim 14, wherein:
said ultrasonic endoscope further includes temperature sensing means for sensing a temperature of said heat transfer material; and
said ultrasonic diagnostic apparatus further comprises temperature control means for controlling an operation of said heat transfer material circulating means based on a sensing result of said temperature sensing means.

17. The ultrasonic diagnostic apparatus according to claim 14, wherein:
said ultrasonic endoscope further includes temperature sensing means for sensing a temperature in the insertion part of said ultrasonic endoscope; and
said ultrasonic diagnostic apparatus further comprises temperature control means for controlling an operation of said heat transfer material circulating means based on a sensing result of said temperature sensing means.
